(19) United States
(12) Patent Application Publication
(10) Pub. No.: US 2003/0088213 A1
(43) Pub. Date: May 8, 2003

(54) Catheter with detachable hub

(76) Inventors: Timothy Schweikert, Levittown, PA (US); John Stephens, Perkiomenville, PA (US)

Correspondence Address: MONTE & MCGRAW, PC, 4092 SKIPPACK PIKE, P.O. BOX 650, SKIPPACK, PA, 19474, US

(21) Appl. No.: 10/271,182

(22) Filed: Oct. 15, 2002

Related U.S. Application Data
(60) Provisional application No. 60/329,593, filed on Oct. 15, 2001.

(51) Int. Cl. A61M 5/32

(52) U.S. Cl. 604/177; 604/533

(57) ABSTRACT

A detachable catheter hub is disclosed. The hub includes a first portion having a proximal end fluidly connectable to at least one catheter extension tube and a distal end fluidly connectable to at least one catheter lumen. The at least one catheter extension tube is fluidly communicable with the at least one catheter lumen. A second portion of the hub is releasably connected to the distal end of the first portion of the hub such that the second portion is adapted to bias the at least one catheter lumen against the distal end of the first portion of the hub. A catheter assembly incorporating the hub is also disclosed. A method of inserting the catheter assembly into a patient is also disclosed.

### CROSS REFERENCE TO RELATED APPLICATION

The present application claims priority from U.S. Provisional Patent Application Serial No. 60/329,593 filed Oct. 15, 2001.

### BACKGROUND OF THE INVENTION

Catheters for the introduction or removal of fluids may be located in various venous locations and cavities throughout the body for introduction or removal of these fluids. Such catheterization may be performed by using a single catheter having multiple lumens. A typical example of a multiple lumen catheter is a dual lumen catheter in which one lumen introduces fluid and the other lumen removes fluid. Catheterization may also be performed by using separate, single lumen catheters inserted through two different incisions into an area to be catheterized. Such multiple catheter assemblies are known as TESIO® catheters.

Generally, to insert any catheter into a blood vessel, the vessel is identified by aspiration with a long hollow needle in accordance with the well known Seldinger technique. When blood enters a syringe attached to the needle, indicating that the vessel has been found, a thin guide wire is then introduced, typically through a syringe needle or other introducer device into the interior of the vessel. The introducer device is then removed, leaving the guide wire within the vessel. The guide wire projects beyond the surface of the skin. At this point, several options are available to a physician for catheter placement. The simplest is to pass a catheter into the vessel directly over the guide wire. The guide wire is then removed, leaving the catheter in position within the vessel. However, this technique is only possible in cases where the catheter is of a relatively small diameter, made of a stiff material, and not significantly larger than the guide wire, for example, for insertion of small diameter dual lumen catheters. If the catheter to be inserted is significantly larger than the guide wire, a dilator device is passed over the guide wire to enlarge the hole. The catheter is then passed over the guide wire, and the guide wire and dilator are then removed.

For chronic catheterization, in which the catheter is intended to remain inside the patient for extended period of time, such as for weeks or even months, it is typically desired to subcutaneously tunnel the catheter using various tunneling techniques. The catheter is typically tunneled into the patient prior to inserting the catheter into the patient's vein. However, there may be times when it is more advantageous, such as depending on the patient or the implanting surgeon's skill, to perform the tunneling after the catheter is implanted in the patient. For some catheters, though, such as multiple lumen catheters with a hub and with bonded luers on the proximal ends of the catheters, it is impractical to perform the tunneling after the catheter is installed in the patient. It would be beneficial to provide a catheter assembly that provides a surgeon with alternative installation procedures for installing the catheter that better suit either the patient's needs or the surgeon's skills.

Further, for chronically installed catheters, portions of the catheter external to the patient occasionally fail, such as for instance, by leaking and/or by the introduction of foreign particles such as dirt, bacteria, and the like into the catheter, necessitating removal of the entire catheter from the patient. Such failures include worn or broken clamps or broken luers. In order to correct these problems, it is presently necessary to remove the entire catheter from the patient, causing additional trauma to the patient and risking additional medical problems to the patient. It would be beneficial to provide a catheter in which the proximal portion of the catheter may be removed and replaced without disturbing the distal portion of the catheter inside the patient.

### BRIEF SUMMARY OF THE INVENTION

In accordance with the invention, a detachable catheter hub is provided. The hub comprises a first portion having a proximal end fluidly connectable to at least one catheter extension tube and a distal end fluidly connectable to at least one catheter lumen such that the at least one catheter extension is fluidly communicable with the at least one catheter lumen. A second portion of the hub is releasably connected to the distal end of the first portion such that the second portion is adapted to bias the at least one catheter lumen against the distal end of the first portion.

Further, a catheter assembly is provided. The catheter assembly comprises at least one catheter extension tube having a proximal end and a distal end and a hub fluidly connected to the distal end of the catheter extension tube. The hub has a first portion and a second portion releasably connected to the first portion. At least one catheter lumen is releasably connected to the first portion of the hub and fluidly connected to the at least one catheter extension tube. The second portion of the hub is releasably engaged with a proximal end of the at least one catheter lumen and biases the at least one catheter lumen against the first portion of the hub.

A method of replacing a hub on a catheter is also provided. The method comprises providing a catheter having at least one catheter extension tube at a distal end of the catheter, at least one catheter lumen at a proximal end of the catheter, and a catheter hub fluidly connecting the at least one catheter extension tube to the at least one catheter lumen. The catheter hub comprises an old first portion having a proximal end connected to the at least one catheter extension tube and a distal end releasably connected to the at least one catheter lumen, and a second portion releasably connected to the old first portion, wherein the second portion biases the at least one catheter lumen against the old first portion. The method further comprises separating the old first portion of the hub from the second portion; separating the old first portion of the hub from the at least one catheter lumen; installing a new first portion of the hub on the at least one catheter portion; and securing the second portion of the hub to the new first portion of the hub.

The present invention further provides a method of inserting a catheter into a patient. The method comprises making an incision through a patient's skin; forming a subcutaneous tunnel under the patient's skin proximate to the incision; inserting a distal end of a catheter into the incision; inserting a proximal end of the catheter through the tunnel away from the incision; connecting a catheter hub to the proximal end of the catheter; and closing the incision.

### BRIEF DESCRIPTION OF THE DRAWINGS

The foregoing summary, as well as the following detailed description of preferred embodiments of the invention, will be better understood when read in conjunction with the appended drawings, which are incorporate herein and constitute part of this specification. For the purpose of illustrating the invention, there are shown in the drawings embodiments which are presently preferred. It should be understood, however, that the invention is not limited to the precise arrangements and instrumentalities shown. In the drawings, the same reference numerals are employed for designating the same elements throughout the several figures. In the drawings:

FIG. 1 is a side view, partially in section, of a catheter assembly incorporating a detachable hub according to a presently preferred embodiment of the invention.

FIG. 2 is an exploded side view of the catheter assembly with detachable hub as shown in FIG. 1.

FIG. 3 is an exploded perspective view of the catheter assembly with detachable hub as shown in FIG. 1.

FIG. 4 is a sectional view of catheter lumens taken along line 4-4 of FIG. 2.

FIG. 5 is a sectional view of a hub body taken along line 5-5 of FIG. 2.

FIG. 6 is an end view of a catheter hub cover with rotatable suture wing according to an embodiment of the present invention.

FIG. 7 is a sectional view of the catheter hub cover and rotatable suture wing taken along line 7-7 of FIG. 6.

FIG. 8 is a sectional view of a catheter hub body according to an alternate embodiment of the present invention.

FIG. 9 is a sectional view of the hub cover partially threaded onto the hub body of the catheter hub according to an embodiment of the present invention.

FIG. 10 is a sectional vie of the hub cover filly threaded onto the hub body of the catheter hub according to an embodiment of the present invention.

FIG. 11 is a view of the catheter assembly according to the present invention inserted into a patient in accordance with one method of inserting the catheter assembly into the patient according to the present invention.

### DETAILED DESCRIPTION OF THE INVENTION

In the drawings, like numerals indicate like elements throughout. Certain terminology is used herein for convenience only and is not to be taken as a limitation on the present invention. The words “proximal” and “distal” refer to directions away from and closer to, respectively, the insertion tip of the catheter in the catheter assembly according to the present invention. The terminology includes the words above specifically mentioned, derivative thereof, and words of similar import. The following describes a preferred embodiment of the invention. However, it should be understood based on this disclosure, that the invention is not limited by the preferred embodiment described herein.

Referring now to the drawings in detail, there is shown in FIGS. 1-3 a catheter assembly indicated generally as 100. The catheter assembly 100 shown in FIGS. 1-3 is a double lumen assembly, although those skilled in the art will recognize that a single lumen assembly or a catheter assembly with more than two lumens may be used within the spirit and scope of the present invention.

The catheter assembly 100 of the present invention may be adapted for use in various applications in which bodily fluids, medicaments or other solutions are introduced into and removed from the body such as perfusion, infusion, plasmapheresis, hemodialysis, chemotherapy, and the like. The area to be catheterized is preferably a blood vessel such as an internal jugular vein, but may be any suitable area within the body. Other areas in which the catheter assembly may be used include, for example, other blood vessels, including the femoral and subclavian veins, any abscess cavity, postoperative cavity, the peritoneal cavity, and other areas of the body including intra-abdominal, sub-diaphragmatic and sub-hepatic areas. It should be understood by one of ordinary skill in the art from this disclosure that these areas are exemplary, and that the catheter assembly may be used to remove or introduce fluids in various areas to be catheterized.

The preferred embodiment of the invention as shown in FIGS. 1-3 is preferably useful for intake, or removal, of blood to be purified from a blood vessel, such as the internal jugular vein, and introduction of purified blood into the same vessel. The blood can be purified by any suitable hemodialysis apparatus (not shown) attached in communication with lumens of the catheter assembly 100 of the invention. The catheter assembly 100 may also be used to introduce medication or other fluids, including, for example, glucose or saline solutions into the patient's body.

For the purposes of describing the preferred embodiment of the present invention, the catheter assembly 100 will be described with respect to the preferred application of hemodialysis, more specifically, for purifying blood flowing through the internal jugular vein. However, it will be understood by one skilled in the art based on this disclosure, that the catheter assembly 100 may be configured and adapted, by increasing or decreasing the catheter size and/or number of catheters and/or lumens in the catheter assembly 100, such that the catheter assembly 100 may be beneficially used for other medical applications in which fluids are introduced into and/or removed from the body.

The catheter assembly 100 is generally constructed from a catheter 120, comprising a distal portion of the catheter assembly 100, an extension portion 130, comprising a proximal portion of the catheter assembly 100, and a hub 150, which releasably connects the distal portion to the proximal portion.

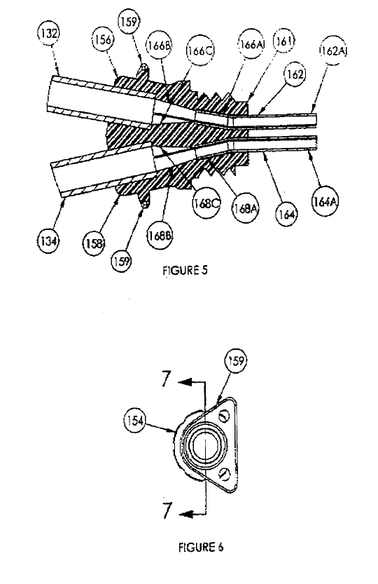

Referring still to FIGS. 1-3, the catheter 120 is comprised of a first lumen 122 and a second lumen 124. The first lumen 122 has a distal portion 122A and a proximal portion 122B and the second lumen 124 has a distal portion 124A and a proximal portion 124B. The distal portions 122A, 124A are configured to be placed, or inserted into, the area to be catheterized, whereas the proximal portions 122B, 124B remain outside of the area. As shown in FIG. 4, each of the first and second lumens 122, 124 is generally “D-shaped” in cross-section, with the flat portion of the first lumen 122 in facing relation with the flat portion of the second lumen 124.

It is preferred that the lumens 122, 124 and respective rounded wall portions and flat side portions are identical to each other so that the catheter 120 has a generally circular cross-section. It should be understood, however, based on this disclosure, that the lumens 122, 124 may be further subdivided and/or additional catheter tubes of the same or varied cross-sectional configuration may be provided within the scope of this invention. For example, those skilled in the art will recognize that the first and second lumens 122, 124 may each have a generally circular cross-sectional area. Further, more than two lumens 122, 124 may be used. Further, although two separate lumens 122, 124 are shown and described, those skilled in the art will recognize that a single lumen with a septum dividing the lumen into separate conduits may be used.

While the generally semi-circular cross section as shown in FIG. 4 is the preferred configuration for fluid flow in each lumen 122, 124, other configurations may be used without departing from the spirit of the present invention, such as, for example, oval, circular, elliptical, square, triangular and kidney-bean shaped. The catheter assembly 100, when having such luminal configurations, will have a varied cross section accordingly. The lumens 122, 124 may be of equal cross-sectional or of different cross-sectional areas. For example, a third lumen (not shown) having a small cross sectional area in comparison with the first and second lumens 122, 124 may be used for infusion of medication. The catheter assembly 100 is also not necessarily circular in cross section in the configuration having unequal cross-sectional areas.

Referring back to FIGS. 1-3, the distal portion 122A, 124A of each lumen 122, 124 has at least one, and preferably, a plurality of openings (not shown) for the transfer of fluid between the catheter 100 and a patient, as is well known by those skilled in the art. The proximal portion 122B, 124B of each lumen 122, 124 is releasably connected to the hub 150, as is explained in greater detail herein. Each lumen 122, 124 may be color coded to designate flow direction of fluid inside each lumen 122, 124. For example, the first lumen 122 may be red, to designate arterial flow to the patient, and the second lumen 124 may be blue, to designate venous flow from the patient. Alternatively, color coded or other indicia may be printed on the lumens 122, 124.

The first lumen 122 and the second lumen 124 are both preferably made of a biocompatible plastic or elastomer, more preferably from a biocompatible elastomer. Suitable biocompatible plastics include materials such as, for example, polyethylene, homopolymers and copolymers of vinyl acetate such as ethylene vinyl acetate copolymer, polyvinylchlorides, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyurethanes, polyvinylpyrrolidone, 2-pyrrolidone, polyacrylonitrile butadiene, polycarbonates, polyamides, fluoropolymers such as homopolymers and copolymers of polytetrafluoroethylene and polyvinyl fluoride, polystyrenes, homopolymers and copolymers of styrene acrylonitrile, cellulose acetate, homopolymers and copolymers of acrylonitrile butadiene styrene, polymethylpentene, polysulfones, polyesters, polyimides, polyisobutylene, polymethylstyrene and other similar compounds known to those skilled in the art. It should be understood that these possible biocompatible polymers are included above for exemplary purposes and should not be construed as limiting. If a biocompatible polymeric material is used to form the lumens 122, 124, it is most preferred that the polymeric material includes a polyurethane or a polyolefin polymeric material having a preferably soft durometer, as specified below.

Suitable, preferred, biocompatible elastomers for use in forming the lumens 122, 124 include biocompatible elastomers such as medical grade silicone rubbers, polyvinyl chloride elastomers, polyolefin homopolymeric and copolymeric elastomers, urethane-based elastomers, and natural rubber or other synthetic rubbers. Preferably, the lumens 122, 124 are made of the elastomeric material such that they are flexible, durable, soft, and easily conformable to the shape of the area to be catheterized and minimize risk of harm to vessel walls. If the lumens 122, 124 are used for hemodialysis applications, they are preferably formed of a soft silicone elastomer, which has a hardness of at least about 80-A on a Shore durometer scale. Such an elastomer is available from Dow Corning, and may include 20% barium sulfate in the elastomer to provide radiopacity. While it is preferred to have a higher Shore durometer hardness if a biocompatible elastomer is used, particularly for hemodialysis, it is also possible to make a device from an elastomer having a lower Shore durometer hardness without departing from the spirit of the invention. It will be understood, based on this disclosure, that the lumens 122, 124 may also be radiopaque depending on their intended use.

A fabric cuff 128 is disposed on a portion of the exterior of the lumens 122, 124, generally proximate the proximal portions 122B, 124B of the lumens 122, 124. The portion of the lumens 122, 124 located distal of the cuff 128 are inserted into the patient through an incision during catheterization, and the portion of the lumens 122, 124, as well as the remaining portions of the catheter assembly 100, remain exterior of the incision. The cuff 128 provides a surface for the patient's skin to graft to the catheter assembly 100, sealing the entrance of the lumens 122, 124 into the patient.

The extension portion 130 is preferably comprised of a first extension tube 132 having a distal portion 132A and a proximal portion 132B, and a second extension tube 134 having a distal portion 134A and a proximal portion 134B. The proximal portion 132B of the first extension tube 132 is connected to a connecting device, such as a luer 136. The proximal portion 134B of the second extension tube 134 is connected to another connecting device, such as a luer 138. The luers 136, 138 are releasably connectable to a device exterior to the catheter assembly 100, such as a dialysis machine (not shown). When the catheter assembly 100 is not in use and is disconnected from the dialysis machine, each luer 136, 138 may be secured by a locking device, not shown, to prevent foreign material from entering into either of the extension tubes 132, 134, and to prevent blood from leaking out of the patient through either of the extension tubes 132, 134.

The distal portion 132A of the first extension tube 132 and the distal portion 134A of the second extension tube 134A are each connected to the hub 150, as is described in more detail herein. A catheter clamp 140, 142, which is well known in the art, may be connected to each extension tube 132, 134 between each luer 136, 138 and the hub 150, respectively. The catheter clamps 140, 142 pinch off flow through the extension tubes 132, 134 during periods of non-use. Typically, each catheter clamp 140, 142 is color coded to its respective luer 136, 138. For example, the catheter clamp 140 and its respective luer 132 may be red, to designate arterial flow to the patient, and the catheter clamp 142 and its respective luer 138 may be blue, to designate venous flow from the patient. The color code matches the color coding on the lumens 122, 124 to ensure that the proper lumens 122, 124 are fluidly connected to their respective extension tubes 132, 134. Although color coding is preferred, those skilled in the art will recognize that other coding systems may be used.

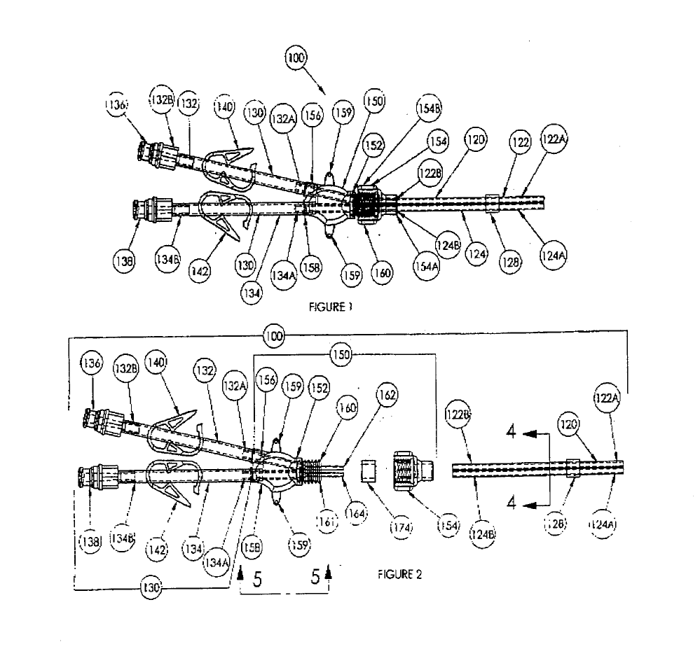

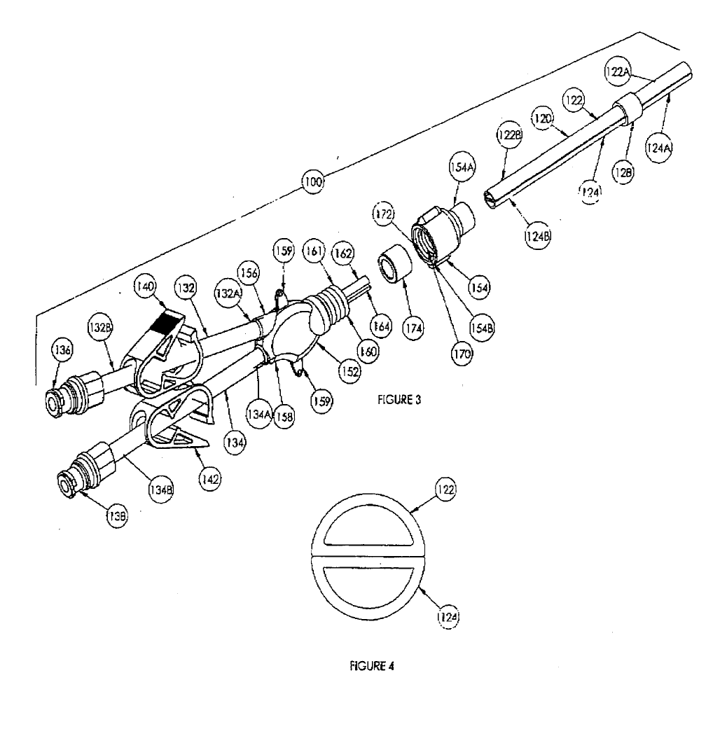

Referring now also to FIG. 5, the hub 150 is comprised of a proximal portion, such as a hub body 152 and a separate distal portion, such as a hub cover 154 that is releasably connected to the hub body 152. The hub body 152 includes an extension tube connection 156, 158 for each respective extension tube 132, 134. While the catheter assembly 100 shown in FIGS. 1 and 2 has two extension tubes 132, 134 and two corresponding extension tube connections 156, 158, those skilled in the art will recognize that, for a catheter having more or less than two extension tubes, a corresponding number of extension tube connections on the hub body 152 is required.

The extension tubes 132, 134 may be fixedly connected to each of their respective extension tube connections 156, 158 by molding the hub body 152 over the distal portions 132A, 134A of the extension tubes 132, 134. Alternatively, the distal portions 132A, 134A of each catheter extension 132, 134 may be secured to the extension tube connections 156, 158 by other means, such as by an adhesive, such as a glue or an epoxy, or by other means known by those skilled in the art.

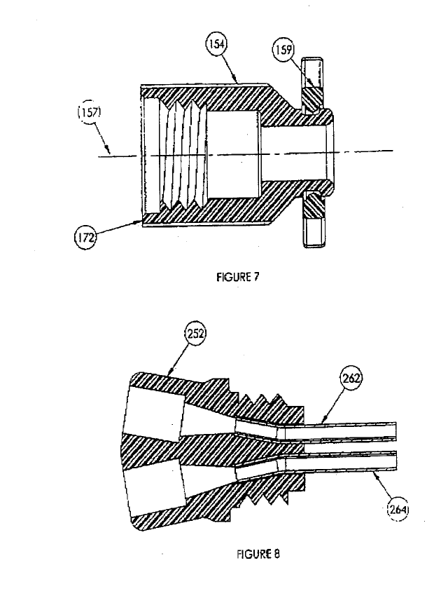

Preferably, the hub body 152 is constructed from pellethane, or any other suitable rigid or semi-rigid material, as will be recognized by those skilled in the art. The hub body 152 preferably includes at least one suture wing 159 which extends away from the hub body 152. The suture wing 159 provides a location on the catheter assembly 100 at which the catheter assembly 100 may be sutured to the skin of the patient to retain the catheter assembly 100 in place. FIGS. 1-3, and 5 show a suture wing 159 with two openings, although those skilled in the art will recognize that a suture wing having more or less than openings may be used. The suture wing 159 may be fixedly connected to the hub body 152 or, alternatively, as shown in the embodiment featured in FIGS. 6 and 7, the suture wing 159 may be rotatably connected to the hub cover 154 so as to rotate about a longitudinal axis 157 of the hub cover 152, allowing more flexibility for the suture wing 159 relative to the hub body 152, as well as allowing the hub cover 154 to be separated from the hub body 152 for the replacement of the proximal portion of the catheter assembly 100 (e.g. the luers 136, 138, the extension tubes 132, 134, and the hub body 152) without the need to remove the sutures from the patient. Although not shown, a generally flexible overmold may be molded over the hub body 152 to provide a soft cover, with the suture wing 159 molded into the overmold. The overmold may be constructed from a thermoplastic elastomer or other known flexible material.

The hub body 152 preferably includes an external threaded connection 160 at a distal end of the hub body 152. A boss 161 is disposed distally of the threaded connection 162. At least one hub cannula 162 extends from the distal end of the hub body 152. In the embodiment shown in FIGS. 1-3, and 5, two hub cannulae 162, 164 extend from the distal end of the hub body 152. The first hub cannula 162 is fluidly connected through the hub body 152 to the first extension tube connection 156 and the second hub cannula 164 is fluidly connected to the second extension tube connection 158. Each of the first and second hub cannulae 162, 164 are preferably constructed from stainless steel, although those skilled in the art will recognize that other materials may be used. In an alternate embodiment, shown in cross-section in FIG. 8, hub cannulae 262, 264 are integrally formed in a hub body 252 from the same material as the hub body. The hub cannulae 262, 264 are molded into the hub body 252 according to known methods to form a single piece.

Referring back to FIG. 5, preferably, each hub cannula 162, 164 has a generally “D-shaped” cross-section that is sized to allow the proximal portion 122B, 124B of each of the first and second lumens 122, 124 to be disposed over the exterior of a respective hub cannula 162, 164 so that each of the first and second lumens 122, 124 conforms to the outer perimeter of the respective hub cannula 162, 164 in a press fit, forming a generally leakproof seal.

Preferably, at least one hub cannula 162, 164 is bent along its length such that distal portions 162A, 164A of the hub cannulae 162, 164 are generally parallel and in close proximity with each other, with the flat portions of each “D-shape” facing each other. Proximal portions 162B, 164B of the hub cannulae 162, 164 are angled away from each other, providing separation between the distal portions 132A, 134A of the extension tubes 132, 134 to facilitate manufacture of the catheter assembly 100. Such an arrangement supports manufacture of a catheter assembly 100 wherein the lumens 122, 124 have “D-shaped” cross-sections and are in close contact with each other, such as in the ASH SPLIT-CATH® catheter, manufactured by Medical Components, Inc. of Harleysville, Pa. Preferably the proximal portions 162B, 164B are angled at approximately 20 degrees with respect to each other, although those skilled in the art will recognize that the angle between the proximal portions 162B, 164B may be other than 20 degrees.

Alternatively, although not shown here, the distal portions 162A, 164A of the hub cannulae 162, 164 may be separated from each other, to facilitate lumens that are either not “D-shaped” in cross-section or not in close contact with each other. Alternatively, each hub cannula 162, 164 may have a generally circular cross-section to facilitate a transition between the extension tubes 132, 134 and lumens (not shown) having circular cross-sections.

Referring still to FIG. 5, to facilitate a transition between a circular cross-section of each extension tube 132, 134 to the “D-shaped” cross-section of each hub cannula, 162, 164, transition portions 166, 168 are formed in the hub 150. The transition portion 166 fluidly connects the extension tube 132 to the hub cannula 162 and the transition portion 168 fluidly connects the extension tube 134 to the hub cannula 164. Each transition portion 166, 168 tapers from a proximal circular cross-section, such as in each of the extension portions 132, 134, to a distal “D-shaped” cross-section, such as in each hub cannula 162, 164.

Referring back to FIG. 3, the hub cover 154 includes a generally circular channel 170 extending through the hub cover between a distal portion 154A and a proximal portion 154B. Preferably, the inner diameter of the channel 170 is larger at the proximal portion 154B, and smaller at the distal portion 154A. The channel 170 may taper down from the proximal portion 154B to the distal portion 154A, or the inner diameter of the channel 170 may change in a step function. The proximal portion 154B includes an internal threaded connection 172 that is sized to threadingly engage the external threaded connection 160 on the hub body 152. Preferably, the hub cover 154 is constructed from an ABS/nylon blend or pellethane, although those skilled in the art will recognize that other suitable materials may be used.

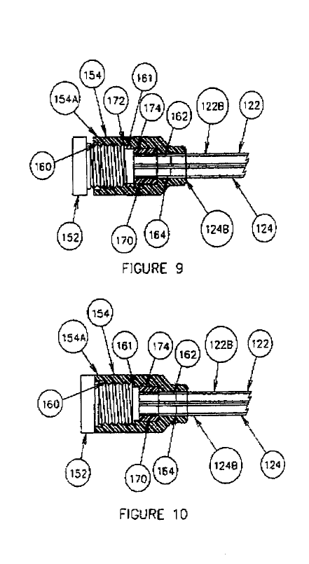

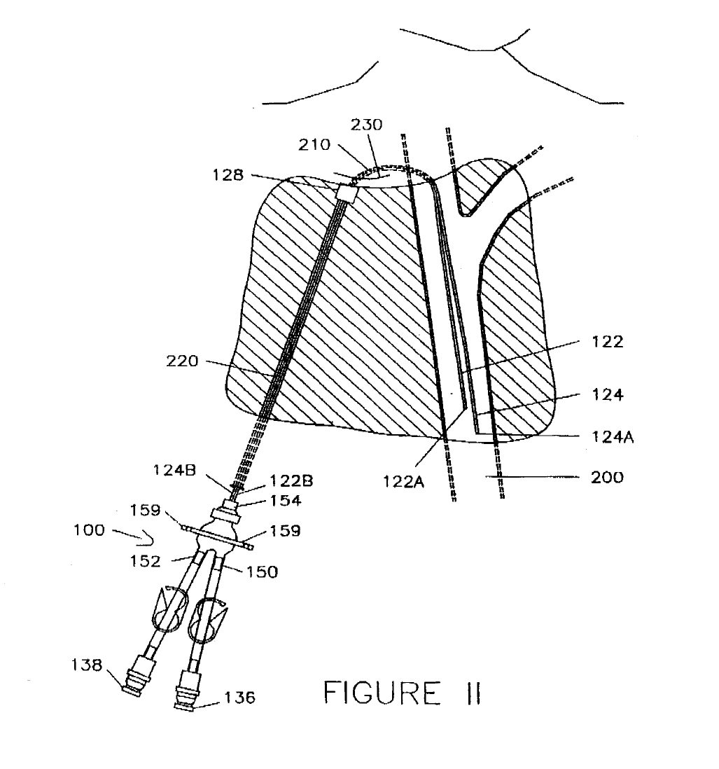

Referring now to FIGS. 9 and 10, the distal portion 154A of the hub cover 154 engages the proximal portions 122B, 124B of the lumens 122, 124 and biases the proximal portions 122B, 124B against the respective hub cannulae 162, 164, further retaining the lumens 122, 124 on the respective hub cannulae 162, 164. Optionally, as shown in FIGS. 9 and 10, a compression ring 174 is disposed within the hub cover 154 between the hub cover 154 and the proximal portions 122B, 124B of the lumens 122, 124. The compression ring 174 has a diameter less than the diameter of the channel 170 at the proximal portion 154B but larger than the diameter of the channel 170 at the distal portion 154A. As a result, when the hub cover 154 is engaged with the threaded connection 160 of the hub body 152, the compression ring 174 is forced by the hub cover 154 over the proximal portions 122B, 124B of the lumens 122, 124 and the distal portions 162A, 164A of the hub cannulae 162, 164, further biasing the proximal portions 122B, 124B of the lumens 122, 124 against the hub cannulae 162, 164. The compression ring 174 is forced by the hub cover 154 to engage the boss 161. The boss 161 prevents the compression ring 174 from engaging the threaded connection 160 on the hub body 152 or the threaded connection 172 on the hub cover 154. This arrangement maintains desired compressive forces on the lumens 122, 124 over the hub cannulae 162, 164 and keeps the torque required to fully engage the hub cover 154 with the hub body 152 to a desired amount.

Although a threaded connection between the hub body 152 and the hub cover 154 is preferred, those skilled in the art will recognize that other methods of releasably connecting the hub body 152 to the hub cover 154, such as, for example, a locking tab and mating slot (not shown), may be used.

Referring now to FIG. 11, in use, the catheter assembly 100 is surgically inserted into the patient's vein 200 to be catheterized through an incision according to methods known in the art. In one method, an insertion site 210 is first determined according to known techniques. Next, a subcutaneous tunnel 220 is formed under the skin proximate to the insertion site 210. The distal portions 122A, 124A of the lumens 122, 124 are then inserted through the tunnel 220 toward the insertion site 210. An incision 230 is next made at the insertion site 210 and the distal portions 122A, 124A of the lumens 122, 124 are inserted through the incision 230 and into the vein 200 to be catheterized, up to the cuff 128. Next, the incision 230 is closed and the hub 150 is secured to an external surface of the body by suturing the adhesive wing 159 to the patient's skin. The open portions of the luers 136, 138 are connected in fluid communication to respective fluid inlets and outlets of a hemodialysis unit, or other fluid transfer equipment (not shown) and dialysis may now begin.

Alternatively, after identification of the insertion site 210, an incision 230 is made in the patient. The subcutaneous tunnel 220 is formed under the skin proximate to the insertion site. The hub cover 154 is removed from the hub body 152 and the proximal portions 122B, 124B of the lumens 122, 124 are separated from the hub cannulae 162, 164, respectively. Next, the compression ring 174 (if used) and the hub cover 154 are removed from the lumens 122, 124. Next, the distal portions 122A, 124A of the lumens 122, 124 are inserted through the incision 230 and into the vein 200 to be catheterized. The proximal portions 122B, 124B of the lumens 122 124 are then inserted into and advanced through the tunnel 220 away from the insertion site 210. The hub cover 154 and the compression ring 174 (if used) are inserted over the proximal portions 122B, 124B of the lumens 122, 124, and the proximal portions 122B, 124B of the lumens are fluidly connected to the hub cannulae 162, 164, respectively. The hub cover 154 is then connected to the hub body 152, preferably by threading the hub cover 154 onto the threads 160 on the hub body 152. Next, the hub 150 may be sutured to the patient's skin via the suture wings 159. The open ends of the luers 136, 138 are connected in fluid communication to respective fluid inlets and outlets of a hemodialysis unit, or other fluid transfer equipment (not shown) and dialysis may now begin.

If during the time that the catheter assembly 100 is in the patient, a problem or malfunction occurs with the distal portion of the catheter assembly 100, such as with either of extension tubes 132, 134, the luers 136, 138, or the clamps 140, 142, the hub cover 154 may be disconnected from the hub body 152 and the problem or malfunction may be corrected without having to take the entire catheter assembly 100 out of the patient.

The hub cover 154 is unthreaded from the hub body 152 and the hub cover 154, along with the compression ring 174 (if used), is slid down the lumens 122, 124 toward the proximal portions 122A, 124A of the lumens 122, 124, exposing the connection between the lumens 122, 124 and the respective hub cannulae 162, 164. The hub cannulae 162, 164 are removed from the lumens 122, 124, thus separating the hub body 152 from the lumens 122, 124. The proximal portion of the catheter assembly 100, extending from the luers 136, 138 to the hub body 152, is removed, and a new proximal portion of the catheter assembly 100, extending from the luers 136, 138 to the hub body 152, is connected to the remaining distal portion of the catheter assembly 100. Optionally, the hub cover 154 may also be removed from the proximal portion of the catheter assembly 100.

Regarding installation of the new proximal portion of the catheter assembly 100, if the original hub cover 154 was removed from the catheter assembly 100, a new hub cover 154 is installed over the proximal portion 122B, 124B of the lumens 122, 124 such that the distal portion 154A of the hub cover 154 is disposed closer to cuff 128 than the proximal portion 154B of the hub cover 154. If a compression ring 174 is used, the compression ring 174 is disposed over the proximal portions 122B, 124B of the lumens 122, 124.

Next, each hub cannula 162, 164 is inserted into a respective lumen 122, 124 such that, the hub cannula 162, 164 that corresponds to a particular extension tube 132, 134 is inserted into the proper lumen 122, 124. The color coding, or whatever other coding system is used, is used to identify the particular extension tube 132, 134 associated with a particular lumen 122, 124 so that the proper fluid connections are made. The open ends of the luers 136, 138 are connected in fluid communication to respective fluid inlets and outlets of a hemodialysis unit, or other fluid transfer equipment (not shown) and dialysis may now begin.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A detachable catheter hub comprising:
a first portion having:
a proximal end fluidly connectable to at least one catheter extension tube; and
a distal end fluidly connectable to at least one catheter lumen such that the at least one catheter extension is fluidly communicable with the at least one catheter lumen; and
a second portion releasably connected to the distal end of the first portion such that the second portion is adapted to bias the at least one catheter lumen against the distal end of the first portion.

2. The detachable catheter hub according to claim 1, wherein the first portion has a threaded connection proximate to the distal end and the second portion has a mating threaded connection.

3. The detachable catheter hub according to claim 1, further comprising a compression ring disposed within the second portion of the hub, wherein the second portion of the hub is adapted to bias the compression ring against the catheter lumen.

4. The detachable hub according to claim 3, wherein the distal end of the first portion comprises a threaded connection and a boss disposed distal of the threaded connection, such that, when the second portion is releasably connected to the first portion, the boss retains the compression ring away from the threaded connection.

5. The detachable catheter hub according to claim 1, wherein the first portion of the hub further comprises at least one suture wing extending therefrom.

6. The detachable catheter hub according to claim 1, wherein the second portion of the hub further comprises at least one suture wing extending therefrom.

7. The detachable catheter hub according to claim 1, wherein the hub further comprises a hub cannula extendable into the at least one catheter lumen.

8. The detachable catheter hub according to claim 1, further comprising a suture wing rotatably connected to the second portion and rotatable about a longitudinal axis of the second portion.

9. The detachable hub according to claim 8, wherein the suture wing comprises a plurality of suture openings.

10. The detachable hub according to claim 1, wherein the first portion further comprises at least one cannula fluidly connecting the distal end to the proximal end, wherein the at least one cannula is integrally formed with the first portion.

11. The detachable hub according to claim 10, wherein the first portion and the at least one cannula are a single piece.

12. A catheter assembly comprising:
at least one catheter extension tube having a proximal end and a distal end;
a hub fluidly connected to the distal end of the catheter extension tube, wherein the hub has a first portion and a second portion releasably connected to the first portion; and
at least one catheter lumen releasably connected to the first portion of the hub and fluidly connected to the at least one catheter extension tube, wherein the second portion of the hub is releasably engaged with a proximal end of the at least one catheter lumen and biases the at least one catheter lumen against the first portion of the hub.

13. The catheter assembly according to claim 12, wherein the hub further comprises at least one insert extending into the proximal end of the at least one lumen.

14. The catheter assembly according to claim 12, further comprising a compression ring disposed within the second portion of the hub, wherein the second portion of the hub biases the compression ring against the proximal end of the lumen.

15. The catheter assembly according to claim 12, wherein the first portion of the hub comprises a threaded connection and the second portion of the hub comprises a mating threaded connection.

16. The catheter assembly according to claim 12, wherein the first portion of the hub further comprises at least one suture wing extending therefrom.

17. The catheter assembly according to claim 12, wherein the secpnd portion of the hub further comprises at least one suture wing extending therefrom.

18. The catheter assembly according to claim 12, wherein the at least one catheter lumen comprises a first plurality of catheter lumens and the at least one catheter extension tube comprises a second plurality of extension tubes, such that each of the second plurality of extension tubes is fluidly connected to at least one of the first plurality of catheter lumens.

19. The catheter assembly according to claim 18, wherein each of the plurality of catheter lumens is coded to identify the catheter extension tube fluidly connected to each of the plurality of catheter lumens according to a code.

20. The catheter assembly according to claim 19, wherein the code is a color code.

21. The catheter assembly according to claim 20, wherein the first plurality of catheter lumens comprises a first catheter lumen releasably connected to a second catheter lumen along a longitudinal plane between the first and second catheters.

22. A method of replacing a hub on a catheter comprising:
providing a catheter having:
at least one catheter extension tube at a proximal end of the catheter;
at least one catheter lumen at a proximal end of the catheter; and
a catheter hub fluidly connecting the at least one catheter extension tube to the at least one catheter lumen, wherein the catheter hub comprises:
an old first portion having a proximal end connected to the at least one catheter extension tube and a distal end releasably connected to the at least one catheter lumen; and
a second portion releasably connected to the old first portion, wherein the second portion biases the at least one catheter lumen against the old first portion;
separating the old first portion of the hub from the second portion;
separating the old first portion of the hub from the at least one catheter lumen;
installing a new first portion of the hub on the at least one catheter lumen; and
securing the second portion of the hub to the new first portion of the hub.

23. The method according to claim 20, further comprising, after separating the old first portion of the hub from the at least one catheter lumen, removing the second portion of the hub from the at least one catheter lumen and, prior to installing the new first portion of the hub on the at least one catheter lumen, inserting a new second portion of the hub over the at least one catheter portion, and wherein securing the second portion of the hub to the new first portion of the hub comprises securing the new second portion of the hub to the new first portion of the hub.

24. A method of inserting a catheter into a patient comprising:
making an incision through a patient's skin;
forming a subcutaneous tunnel under the patient's skin proximate to the incision;
inserting a distal end of a catheter into the incision;
inserting a proximal end of the catheter through the tunnel away from the incision;
connecting a catheter hub to the proximal end of the catheter; and
closing the incision.

25. The method according to claim 22, further comprising, after connecting the proximal end of the catheter to the catheter hub, securing the catheter hub to the patient's skin.
